(19) United States
(12) Patent Application Publication
(10) Pub. No.: US 2025/0017506 A1
(43) Pub. Date: Jan. 16, 2025

(54) ARM-WORN ELECTROCARDIOGRAPHIC DEVICE AND METHOD FOR ELECTROCARDIOGRAPHIC SIGNAL MEASUREMENT

(71) Applicant: DEMEDAR SPOLKA Z OGRANICZONA ODPOWIEDZIALNOSCIA, Wroclaw (PL)

(72) Inventors: Ryszard KLEMPOUS, Wroclaw (PL); Jan NIKODEM, Wroclaw (PL); Konrad KLUWAK, Wroclaw (PL); Dariusz JAGIELSKI, Sadkow (PL)

(21) Appl. No.: 18/714,448

(22) PCT Filed: Nov. 29, 2022
(86) PCT No.: PCT/PL2022/050086

§ 371 Date: May 29, 2024

(30) Foreign Application Priority Data
Nov. 29, 2021 (PL) P.439675

(51) Int. Cl. A61B 5/256; A61B 5/00; A61B 5/282; A61B 5/318

(52) U.S. Cl. CPC A61B 5/256; A61B 5/282; A61B 5/318; A61B 5/6824; A61B 5/6831

(57) ABSTRACT

The first object of the invention is an arm-worn electrocardiographic device in a shape of a band, comprising a measurement module for processing the measured electrocardiographic signal, characterised in that the first measuring wing and the second measuring wing are attached to the measurement module, symmetrically with respect to each other, by means of spring connectors, for measuring the electrocardiographic signal, and a first fastening strip and a second fastening strip are attached to each of the measuring wings, respectively, by means of spring connectors, wherein the measurement module, the measuring wings, and the fastening strips have an inner surface for contacting the user's skin and an outer surface for fixing the personalizing attachment, and on the inner surface of the module a third electrode is located, on the inner surface of the first measuring wing a first second electrode is located, and on the inner surface of the second measuring wing a second third electrode is located, wherein each of the electrodes comprises a matrix of movable sensors, respectively, wherein the surface area ratio of each electrode to the surface area of the inner surface of the first fastening strip or the second fastening strip is not greater than one, wherein the movable sensor is comprised of a conductive material bonded to an elastic substrate. The invention also relates to a method for electrocardiographic signal measurement.

The object of the invention is an arm-worn electrocardiographic device, in the shape of a band, for a non-invasive recording of electrocardiographic signals performed to obtain a graphic representation of electrical activity of the heart. The device allows detecting cardiac activity disorders (e.g., arrhythmia) both during resting, movement and long-term activity in all everyday life situations. The device is located on the arm which, unlike other possible locations (e.g., on the wrist), allows obtaining an ECG signal with a quality that provides analysing cardiac activity, convenience of use, and long-term recording. The invention also relates to a method for electrocardiographic signal measurement.

From the Polish Patent Application P.422993, a wearable device is known, used for diagnosing the mental state of individual persons and groups of people. The device comprises a module for inputting the assessment of mental state by the subject, an optical indicator, a real-time clock, at least one sensor measuring the biometric parameter of the subject, a control system with a communication assembly for communicating with a mobile telecommunication device, a memory, and an autonomous power supply system. The module for inputting the assessment of mental state is provided with at least one touch- and/or push-activated field, for inputting the mental state, positive or negative. The device is in a form of a headband.

European Patent EP1195134B1 describes a wrist-worn device comprising a display showing a heart rate parameter value, such as a measured heart rate or a heart rate variable derived from the heart rate. ‘The display comprises at least two display areas, the display areas of the wrist-worn device are configured to show that the heart rate parameter momentarily belongs to a heart rate parameter value range corresponding to the display area and the wrist-worn device comprises at least one sliding means for selecting the desired heart rate parameter value range by mechanically sliding the sliding means to cover at least one display area at a time.’ American Patent Application US2019059752 describes a device for measuring the value of blood pressure on a user's limb, e.g. on an arm. The device, taking a form of a measuring module attached by means of bands to the limb, is comprised of an assembly of ECG electrodes, optical and electronic sensors, a microcontroller for signal processing, a power source, user interface means, and a wireless communication unit. Said sensors are located on the inner side of the device, such that they are in direct contact with the skin. The device is intended for monitoring blood pressure and can be used for medical purposes or self-monitoring in everyday life.

From another American Patent Application US2017127966A1, an ECG signal registering device is known. Said device is a two-element system comprising two wristbands. In order to register the ECG signal, both bands must be brought into electrical contact.

European Patent EP2555676B1 describes a device comprising a housing having at least one sensor. The sensor is located on the inner side of the housing, namely on the side that is intended to be applied against the user's skin. The device also comprises a flexible band connected to the housing. The flexible band is chosen in terms of length and flexibility so that it can be wound around a body part of the user, such as an arm, a leg, or an upper arm. Its function is to keep the housing on the user's skin. The strap has an inner side facing the user's skin when the device is being worn. In addition, the housing of the sensor device is provided with at least one stopper device. It is located on the surface of the housing or on the inner side of the band. The stopper device has a higher coefficient of static friction (compared to that of the human skin) than the said surface of the housing or the band. The stopper device extends along a part of the inner side of the band. The second end of the strip of the stopping element, i.e. the end opposite to the first end, is movable with respect to the band. In other words, the strip is, on one end, connected to the housing in a fixed manner but is movable, over at least part of its length, with respect to the band. Because it is arranged along the first side of the band, i.e., the side that faces the user's skin, the band holds the strip against the body during regular operation, such that high static friction arises between the strip and the body. Because the stopper device is connected to the housing, it can hold the housing in place. However, if the band moves in the longitudinal direction, e.g., due to muscle movement, such movement is not or only weakly transferred to the strip. Maintaining a fixed position of the device does not necessarily provide a constant quality of measurements. It only allows it to continue. However, in the case of a change in the shape of the muscles (e.g. due to movement), free space may be created between the measuring electrode and the skin, which affects the measured signal.

Indian Patent Application IN202141019221 describes a cardiac activity monitoring device in a form of a wristband worn on the wrist of one upper limb. The wristband is a portable device that uses a machine learning algorithm to measure, register, and analyse ECG. The device monitors users experiencing atrial fibrillation in real time by means of an implantable electrocardiograph (ECG) sensor. The implantable sensor is comprised of a Raspberry Pi, a battery, a PPG sensor, and two electrodes. The sensor detects ECG signals from two electrodes and transmits them to a cloud memory for further analysis of the user's ECG. The relevant data are collected by Raspberry Pi, and the data analysis is performed utilizing machine learning algorithms.

Korean Patent Application KR100868073A discloses an armband-type device for biosignal measurement. It comprises an ECG electrode, an analogue ECG circuit, a digital controller, and a Bluetooth transmission module. The ECG electrode is comprised of coated Ag/AgCl electrodes and detects ECG signals from the arm. The analogue ECG circuit amplifies and filters the detected ECG signals and converts the ECG signals to digital signals. The digital controller removes the 60 Hz frequency noise of the power supply and the radio frequencies from the signals transmitted from the analogue ECG circuit. The Bluetooth transmission module transmits signals from the digital controller to the computer, the ultramobile PC computer, or a display device on the wrist.

Taiwanese Patent Description TW602543B describes a system for blood pressure measurement, configured mainly to detect an electrocardiographic signal of the subject by means of an ECG detecting module. The ECG signal measurement is conducted by impedance measurement. The impedance is measured by means of a band located on a limb.

US patent U.S. Ser. No. 10/694,966B1 describes an ergonomically designed wireless wearable smart band pair for continuous ECG monitoring. Said pair comprises primary and secondary smart bands with integrated electrodes, which are provided with switches to enable desired electrodes during data acquisition. When the smart bands are worn around both limbs, the electrodes are in contact with the skin. The primary smart band sets all possible states of the electrode switches and acquires biopotential data from the first wrist, while the secondary smart band simultaneously acquires biopotential data from the second wrist and sends it wirelessly to the primary smart band. The primary smart band processes biopotential data via digital and analogue signal conditioning. It combines information to acquire high-fidelity ECG data per Einthoven's law without need for completing to complete a circuit via leads and/or holding auxiliary electrodes. The primary smart band analyses ECG data in real-time, generates relevant alerts, stores data locally, and wirelessly transmits information to external devices.

Another US Patent Application US2009048526 discloses a device for monitoring the user's heart. It comprises several sensors for measuring changes in an electrical parameter of the user's arm, from which changes in the electrocardiogram, heart rate and/or heart rate variability of the user's heart can be determined. The device also comprises a data processor for determining the electrocardiogram, heart rate and/or heart rate variability based on the electrical parameter. In contrast, the output device informs the user about the electrocardiogram, heart rate and/or heart rate variability. The device is in a form of a single band, in particular, a wristwatch.

Yet another US Patent Application US2015335284A1 describes a wearable system and methods for physiological data measurement from a device worn around a body part of a user, comprising a primary module and a sensor module. The primary module is comprised of a display and a primary computing unit. The sensor module is located spatially with respect to the primary module and above the part of the body part in order to measure one or more physiological features. The primary module is placed in a manner adjustable by the user with respect to the sensor module, such that the sensor module maintains its position above the body part in order to provide enough contact with the body part for precise physiological data measurement, independently from anthropometric size of the body part.

Another US Patent Application US2016360971 discloses a method and systems for monitoring the health status of chronically ill people. An exemplary system comprises a wearable device with sensors, wherein the wearable device is designed to be worn on a user's wrist. The wearable device is configured for continuous collection, by the sensors, of data from the sensors from one location on the user's body. The sensor data is processed to obtain electrocardiogram and photoplethysmogram data. The electrocardiographic and the photoplethysmographic data are analysed to obtain medical parameters associated with chronic illness.

Chinese Patent Application CN108294742A discloses a device in a form of an armband, a filtering amplifier, and a microcomputer with a single chip, wherein the armband comprises a textile electrode and a button with a textile electrode. The textile electrode is used to detect the electrocardiographic signal. The filtering amplifier is connected to the armband by means of the button and is used to obtain the electrocardiographic signal and to amplify and filter the ECG signal.

Another Chinese Patent Application CN109998542A describes a wristband for myoelectric signal acquisition based on a textile electrode. The differential electrodes and the reference electrode are evenly spaced on the wristband. They are located in different parts of the band: near the wrist and the middle of the forearm. The distance between every two differential electrodes does not exceed 30 mm, and if this distance is too large, too many effective frequency components of the myoelectric signal are lost. The differential electrodes are arranged in pairs, the number of pairs is equal to n, and the differential electrodes correspond to four muscle groups associated with movement of the hands. The rectangular reference electrode is located in an inactive muscle region, as much as possible, and the reference electrode is used to provide zero reference voltage for quick stabilisation of measurement signals.

Chinese Utility Model CN211985426U description discloses an ECG signal measuring device in a form of a wristband with replaceable electrodes. Including the body, the sidewall of the main body is rotationally connected to the first connecting belt; blocking openings are formed in a surface of the first connecting belt at equal intervals. The second connecting belt is rotationally connected to the side, away from the first connecting belt, the sidewall of the main body, at the end, away from the main body of the second connecting belt, of the lock, a latch attachment is located, corresponding to the lock opening, and an electrode is located at the back of the main body.

PCT International Patent Application WO2015138734A1 describes a wearable device that wirelessly and automatically acquires and processes signals indicative of a condition of a subject's health. The device is adapted to wirelessly transmit the processed signals and other information to a suitable analytic and/or storage device where the subject's condition can be analysed and/or stored. The signals can be ECG signals. The device, according to the solution, has a form of a chestband and an armband.

The scientific publication ‘Wearable Noncontact Armband for Mobile ECG Monitoring System’ (IEEE Transactions on Biomedical Circuits and Systems, vol. 10, no. 6, pp. 1112-1118, December 2016) describes a system for monitoring heart activity through ECG signals. The system consists of capacitive-coupled electrodes embedded in a band. On the surface of the band, which is in contact with the skin, three flexible electrodes, sewn into the band, are located. The electronic part is comprised of a Bluetooth 4.0 module, an ECG module, and a microcontroller. Bluetooth Low Energy (BLE) has been used as the data transmission protocol.

Another scientific publication, ‘Wearable Armband Device for Daily Life Electrocardiogram Monitoring’ (IEEE Transactions on Biomedical Engineering, vol. 67, no. 12, pp. 3464-3473, December 2020), describes an ECG signal monitor to be worn on an arm during longer periods of time. It has a form of a band recording three channels: an ECG channel, an EMG channel, and a tri-axial accelerometer signal. The ECG signal is recorded using an assembly of three pairs of electrodes, arranged alternately, by measuring the impedance. Contrary to conventional Holter monitors, the band-based ECG device is convenient for long-term daily life monitoring because it uses no obstructive leads and has dry electrodes (no hydrogels), which do not cause skin irritation even after a few days. Nevertheless, it can be easily displaced, affecting the recorded signal's quality. Therefore, principal component analysis (PCA) and normalized least mean squares (NLMS) adaptive filtering were used to reduce the EMG noise from the ECG channels. In addition, an artefact detector and an optimal channel selector were developed based on a support vector machine (SVM) classifier with a radial basis function (RBF) kernel using features related to the ECG signal quality.

Another scientific publication, ‘Influence of Armband Form Factors on Wearable ECG Monitoring Performance’ (IEEE Sensors Journal, vol. 21, no. 9, pp. 11046-11060, May 1, 2021), describes the role of electrode location and contact pressure on the ECG armband performance made of an electronic textile (E-textile) worn on the upper left arm. The band is made of stretchable material, into which dry Ag/AgCl electrodes are pressed and fabricated in a process of printing on a thermoplastic polyurethane. However, long-term use of such a band can cause discomfort due to constant point pressure on the skin and thus can cause irritation or even allergic reactions.

The state of the art shows that many attempts were made to develop a device for ECG signal measurement in a manner convenient to the user, e.g. without additional instruments or the need for medical knowledge. Multi-element systems are also known (e.g. chestbelt-armband or double wristband), as well as one-element systems, e.g. single wristband. Although devices known in the art, although they can be used for longer periods of time, e.g. up to 24 hours, they do not provide constant quality of measurement signal in a longer period of time measured in multi-day or multi-week periods. In addition, the devices known in the art do not provide a constant area and/or point of electrocardiographic signal measurement, which can lead to incorrect results. Another problem associated with ECG signal measurement is a necessity to perform the measurement and/or monitor it for a long time, which may involve discomfort caused by prolonged pressure on the skin while using the devices known in the art.

The technical problem faced by the invention would be providing a band with a structure adapting to a changing geometry of the cross-section of the arm while maintaining a fixed location of the measurement area at the interface between the electrode and the skin, which would lead to stabilisation of impedance and/or minimising the impedance changes at the skin-electrode interface during physical activity in a long period of time, wherein the ECG signal measurement would not require use of any additional interface between the device and the user's body and would be realised without any additional auxiliary devices. Furthermore, considering the state of the art, the problem faced by the invention would also be providing a band with a structure that would minimise discomfort caused by pressure on the skin during long-term use of the band.

The first object of the invention is an arm-worn electrocardiographic device in a shape of a band, comprising a measurement module for processing the measured electrocardiographic signal, characterised in that the first measuring wing and the second measuring wing are attached to the measurement module, symmetrically with respect to each other, by means of spring connectors, for measuring the electrocardiographic signal, and a first fastening strip and a second fastening strip are attached to each of the measuring wings, respectively, by means of spring connectors, wherein the measurement module, the measuring wings, and the fastening strips have an inner surface for contacting the user's skin and an outer surface for fixing the personalizing attachment, and on the inner surface of the module a third electrode is located, on the inner surface of the first measuring wing a first second electrode is located, and on the inner surface of the second measuring wing a second third electrode is located, wherein each of the electrodes comprises a matrix of movable sensors, respectively, wherein the surface area ratio of each electrode to the surface area of the inner surface of the first fastening strip or the second fastening strip is not greater than one, wherein the movable sensor is comprised of a conductive material bonded to an elastic substrate.

In a preferred embodiment of the invention, each of the matrices is comprised of N movable sensors.

In another preferred embodiment of the invention, in the movable sensor, the elastic substrate adheres to the conductive material on the opposite surface of the material with respect to its surface configured for contact with the skin.

In a further preferred embodiment of the invention, the elastic substrate of the electrode is an elastomer. However, equally preferably, the conductive material of the movable sensor is chosen from a group including a metal or a conductive plastic.

In another preferred embodiment of the invention, the first and second measuring wings comprise power batteries, wherein the first or the second measuring wing comprises a magnetic socket for attaching an electrical connector and maintaining mechanical stability of the electrical connection with the device for battery charging, for a proper connection between the plug and the socket.

In another preferred embodiment of the invention, the measurement module for processing the measured electrocardiographic signal comprises a component for measuring the electrocardiographic signal, a microprocessor component comprising an analogue-to-digital converter, and a component for wireless communication with an external device.

In yet another preferred embodiment of the invention, the matrix of movable sensors in an arrangement of k columns and l rows of movable sensors comprises 1 to N movable sensors.

The second object of the invention is a method for electrocardiographic signal measurement by means of the arm-worn electrocardiographic device, as defined in the first object of the invention, including:
a) placing the device on the arm,
b) measuring the electrocardiographic signal, where the electrocardiographic signal measurement is achieved by determining the electrical potential difference in a three-electrode system,
c) processing and analysing the measurement signal in order to identify the features characteristic for cardiac activity, characterised in that the single electrode in the three-electrode system corresponds to any electrode of the arm-worn electrocardiographic device, wherein, in order to obtain the electrical potential difference, a first configuration of the movable sensors in the matrix arrangement of the electrodes is determined by means of a system of a microprocessor measuring module, and a measurement of the electrical potential difference is performed, wherein, if the measurement area corresponding to the selected electrode undergoes shifting during the measurement, then a subsequent configuration of the movable sensors in the matrix arrangement of the electrodes is determined by the microprocessor system of the measurement module, wherein the movable sensor is a conductive material bonded with the elastic substrate, and the measurement of the electrical potential difference is performed, wherein, throughout the measurement, K configurations of movable sensors in a matrix arrangement of the electrodes are determined, in order to provide the electrocardiographic signal, in such a way that the value of impedance in the measurement area corresponding to the subsequent configurations of the movable sensors in the matrix arrangement is stable.

In a preferred embodiment of the invention, the matrix of movable sensors in an arrangement of k columns and l rows of movable sensors comprises 1 to N movable sensors.

In another preferred embodiment of the invention, the configuration of the movable sensors in a matrix arrangement of floating electrodes comprises 1 to N movable sensors, where N corresponds to the maximum number of movable sensors in a matrix arrangement of a particular electrode.

In a further preferred embodiment of the invention, the configuration of movable sensors is determined by the microprocessor system of the measurement module using artificial intelligence algorithms, wherein the configuration of movable sensors in a matrix arrangement of the electrodes is determined based on a measured value of impedance changes in the measurement area.

In yet another preferred embodiment of the invention, the ECG measurement signal is processed in an analogue-to-digital converter and analysed using electrocardiographic software of the arm-worn device, wherein the software includes an aggregating classifier system for categorising a fragment of the electrocardiographic signal, a personalised classifier for classifying the criteria of electrocardiographic signal classification, an artefact detector for filtering out the artefacts from the electrocardiographic signal, and an arrhythmia selector extracting the features characteristic for cardiac activity based on the aggregating and personalised classifiers, preferably cardiac activity abnormalities which include the following, outside the range of accepted normal values: increases and decreases in heart rate, presence of variability of R-R intervals or its absence.

In yet another embodiment of the invention, the ECG signal measurement is performed by an ECG measuring component and sent to the ADC converter, the signal is sent to the artefact detector for identification and filtering out the artefacts, next, independently with respect to each other, based on the detected artefact signal, the personalised classifiers and aggregating classifiers are built in the personalised classifier and in the aggregating classifier, respectively, next, the signal is sent to the arrhythmia selector and the cardiac activity anomalies are extracted from it based on the personalised classifiers and aggregating classifiers, and are transmitted to a mobile application by the component for wireless communication with an external device of the measurement module.

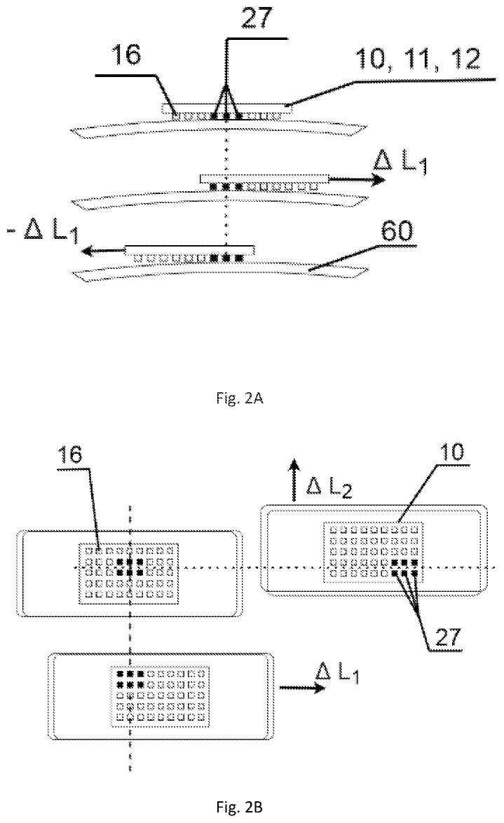

The invention is characterised by a number of advantages. The structure of the band according to the invention provides stabilisation of impedance in the measurement area. This has a beneficial effect on the quality of the recorded signal, obtained by using a matrix of movable sensors, which are bonded to the elastic substrate. This structure provides stabilisation of the compression force F of the electrode against the skin by displacement in and out proportionally to the pressure at a point of contact between the movable sensor and the skin. In addition, the structure of the band, according to the invention, causes that the force with which the movable sensors are pressed to the skin is compensated by elastic elements in the band structure. This leads to decreasing the feeling of discomfort, e.g., caused by long-term, in the order of days or weeks, use of the band. Additionally, a constant measurement quality is provided by the adaptive mechanism of forming a configuration of movable sensors in the measurement matrix/area of the electrode. This mechanism, despite the movements ±ΔL₁ of the band around and the movements ±ΔL₂ of the band along the arm (FIG. 2A-2B), provides stable impedance on the skin-electrode interface, where the ECG signal is measured on the arm.

The embodiments of the invention are depicted in drawings, in which the following are illustrated:

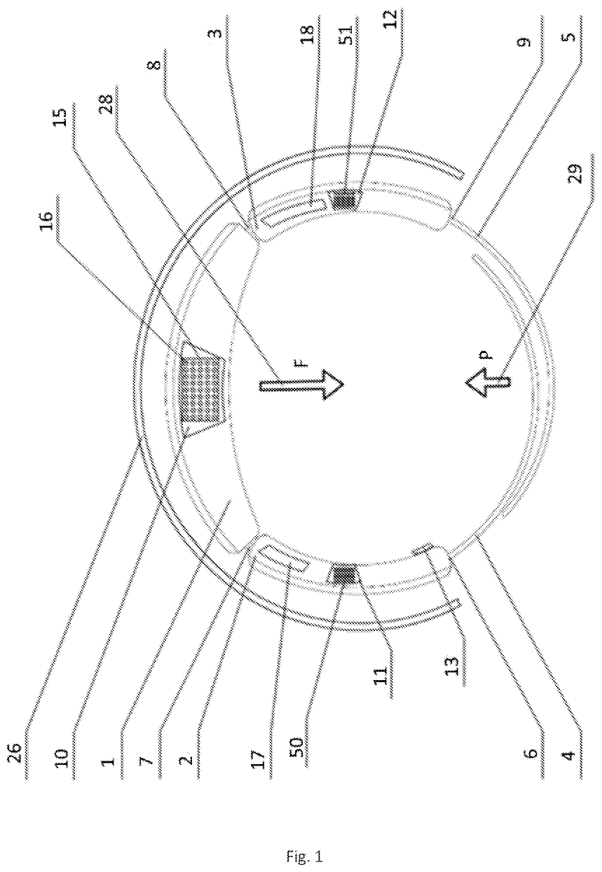

FIG. 1 shows a general view of the band structure,

FIGS. 2A-2B show stabilisation of the contact region between the movable sensors (16) and the skin (60) based on adaptive variable configurations (27) of movable sensors.

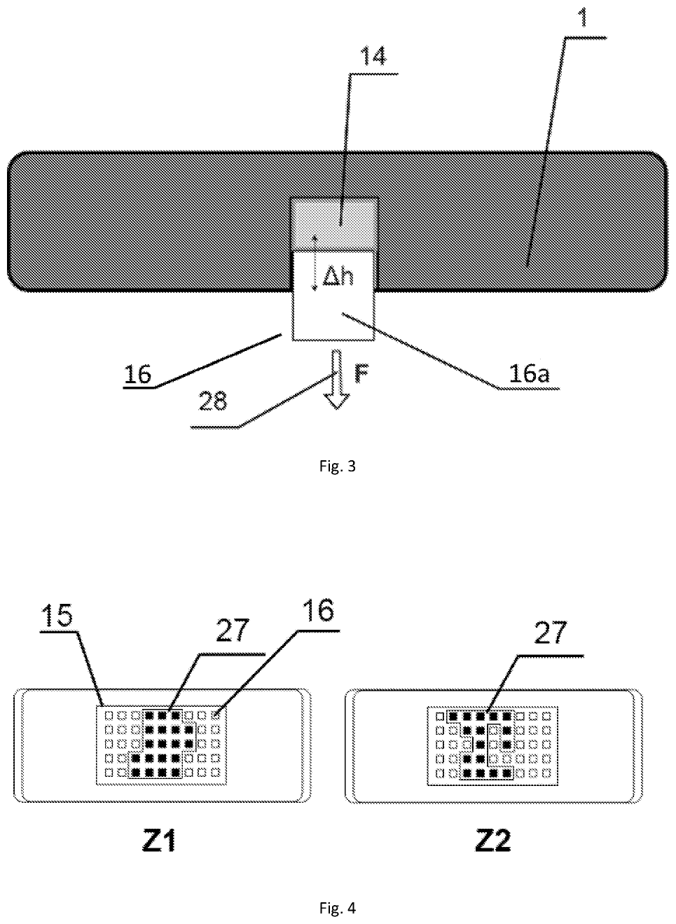

FIG. 3 shows compression force F (28) stabilising system of the movable sensor (16) using the elastic substrate (14),

FIG. 4 shows adaptive forming of an exemplary configuration (27) of movable sensors within the matrix (15),

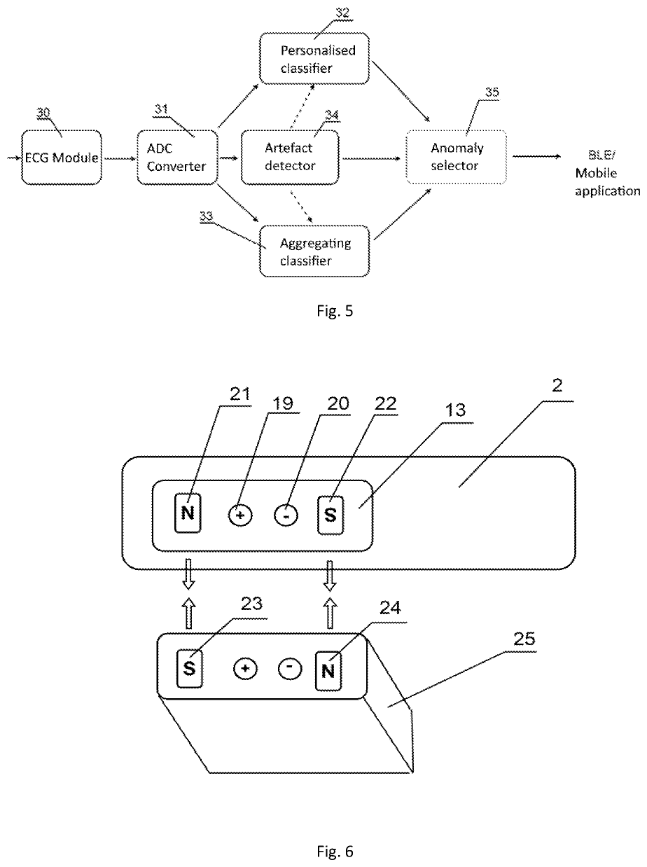

FIG. 5 shows a block diagram of ECG signal processing,

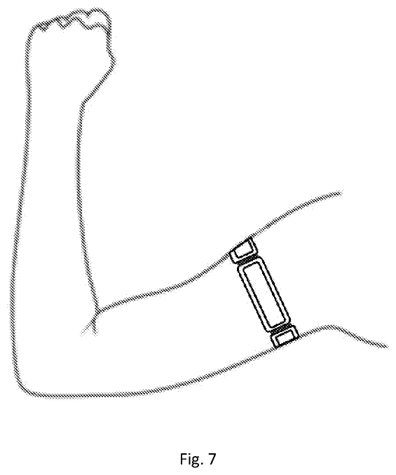

FIG. 6 shows structure of the socket (13)-plug (25) connection of the battery charging system of the band.

### EXAMPLE 1 ARM-WORN ELECTROCARDIOGRAPHIC DEVICE FOR ELECTROCARDIOGRAPHIC SIGNAL RECORDING

The arm-worn electrocardiographic device for ECG signal recording has a form of a band, as shown in FIG. 1. Band is a term used interchangeably with arm-worn electrocardiographic device. The band comprises a measurement module (1), two measuring wings (2) and (3), and fastening strips (4) and (5). The measurement module (1) comprises a component for ECG signal measurement and a microprocessor component comprising an analogue-to-digital converter (ADC). Additionally, it also comprises a component for wireless communication (e.g., a Bluetooth module or a Bluetooth Low Energy module) with an external device (e.g., a mobile phone, a computer). The measuring wings (2) and (3) are attached to the measurement module (1), on both of its sides, symmetrically and opposite, with respect to each other. Further, fastening strips (4) and (5) are attached to the measuring wings (2) and (3); one fastening strip to each of the measuring wings (2) and (3). Individual elements are attached to each other by means of spring connectors (6), (7), (8), (9). Said connectors may be made of a material capable of reversible deformation, e.g., they may be elastic blades or other means for elastically connecting two elements, known in the art. Such structure of the band forces adaptive adjustment of the shape of the band to the geometry of the arm resulting from variable muscle tension, such that the circumferential shape of the band is adjusting its shape to the current cross-section of the arm. This is important for long-term comfort and everyday use of the band. During everyday activities (movement, physical activity), the user's arm is active, i.e., the shape of the arm muscles changes temporarily at the location which is the correct location for placing the band in order to measure the electrocardiographic signal.

Each of the structural elements (1), (2), (3), (4), (5) mentioned above has two surfaces: an outer surface and an inner surface. The inner surface contacts the user's skin while the band is in use. Whereas the outer surface exposes the band in the opposite direction, i.e., towards the user. Additionally, the outer surface is used for fixing the personalizing attachment (26) to the band. The attachment (26) is fixed to the outer surface side of the band by means of snaps and allows the user to individually choose the external appearance of the armband, e.g., pattern and colour. The attachment (26) may be made of metal, polymer, or another material on which a decorative pattern can be applied, or which can be covered with a solid colour using methods known in the art. The attachment (26) has a form of a profiled plate with a width not greater than the transverse dimension of the band according to the invention. Also, the length of the attachment (26) is not greater than the length of the band measured from the ends of the measuring wings (2) and (3) corresponding to the mounting points of the fastening strips (4) and (5). The outer contour of the attachment (26) corresponds to the outer contour of the measurement module and the measuring wings (2) and (3). The attachment (26) has a top and a bottom surface. The top surface of the attachment (26) is the surface preferred for the application of the decorative pattern because it is directly exposed to the environment. Whereas the bottom surface of the attachment (26) is the surface in direct contact with the band, particularly with the measurement module (1) and the measuring wings (2) and (3). The attachment (26) also comprises protrusions for fixing to the band. Fixing of the attachment (26) is carried out by inserting the mounting protrusions into corresponding mounting points located along the outer contour of the measurement module (1) and the measuring wings (2) and (3) of the band.

Additionally, a socket (13) for charging the batteries (17), (18) is located on one of the measuring wings (2), (3), e.g., the wing (2). In an alternative embodiment of the band, the socket (13) may be located on the other measuring wing (3). The socket (13) is comprised of contacts ‘+’ (19) and ‘−’ (20) and a pair of magnets N (21)-S (22), interacting with the magnets S (23)-N (24) located on the plug (25) of the charger (FIG. 6). The charger does not constitute an object of the invention. Using the magnets allows avoiding an improper connection of the plug (25) with the socket (13) and maintains stability of the mechanical connection between them.

However, the batteries (17) and (18) themselves are located inside the measuring wings (2) and (3), respectively.

The measurement module (1) and the measuring wings (2) and (3) comprise electrodes (10), (11), (12) on their surfaces, respectively, which are shown in FIG. 1. Each of said electrodes (10), (11), and (12) has a form of a matrix (15), (50), (51) comprising N movable sensors (16). The structure of movable sensors is shown in FIG. 3. The electrodes (10), (11), (12) are signal elements of the band (1) and are used for reading the ECG signal in a three-electrode system. Each of the electrodes can correspond to only one limb lead (i.e., I, II, or III), as is the case for typical electrocardiographic devices known in the art. The traditional electrodes are based on fixed contact with the skin. Therefore, the user must remain unmoving during measurement. The matrix structure of the electrodes (10), (11), (12), according to the invention, allows active adjustment of their mechanically active elements, i.e., the movable sensors (16) to a changing cross-section geometry of the user's arm, achieved by mounting the movable sensors (16), forming the matrix (15), (50), or (51) of the electrode (10), (11), or (12), on an elastic substrate. Each of the movable sensors (16) can move independently. The term movement should be understood as an ability to adaptively change the position in a 3D space (i.e., radially perpendicularly to the arm, along the arm, and rotationally around the arm) of parameters of the active measurement area of the electrode, providing a stable value of impedance on the skin-electrode interface in the measurement area, where the ECG signal is measured on the arm. In the case of radial movements (perpendicularly to the arm), movement of the movable sensor (16) by a value of Δh (FIG. 3), where the value Δh depends on the pressing force F (28) of a particular movable sensor (16) to the skin surface, provides stabilisation of the pressing force of the movable sensor (16) to the skin. Whereas, in case of movement ±ΔL₁ of the band (1) around or movement ±ΔL₂ of the band (1) along the arm (FIGS. 2A-2B), adjustment of the measurement area is realised by means of adaptive selection of configuration (27) (FIG. 4, FIG. 2B) of the movable sensors (16) in a matrix/measurement area of the electrode (10). This solution facilitates measuring the electrocardiographic signal both during rest and during physical activity of the user.

The electrodes (10), (11), and (12) are comprised of matrices (15), (50), (51), respectively, of movable sensors (16) having a structure shown in FIG. 3, i.e., such that the conductive material (16a), the movable sensor (16) is made of, is bonded with the elastic substrate (14). The elastic substrate may be an elastomeric polymer or other material with similar properties known in the art. Whereas, the conductive material (16a) may be chosen among metals or plastics conducting electrical current or other materials with similar properties known in the art. However, a fundamental requirement is that the system comprised of the conductive material (16a), the binding, and the elastic substrate (14) must provide conduction of the electrical charges, and the conductive material (16a) must be neutral for human skin (e.g., not causing undesirable allergic reactions) and chemically (e.g., not corroding in a high salinity environment). In case of radial movements (perpendicularly to the arm), adjustment of the pressing force F (28) of the movable sensor (16) while pressing it to the skin is performed by compressing the substrate (14). This structure of the movable sensor (16), and further the matrix (15) comprised of multiple movable sensors (16), provides stabilisation of the pressing force F (28) of the electrodes (10), (11), or (12) to the skin, by displacement in and out to a height Δh with respect to the band and proportionally to the force in a point corresponding to pushing a particular movable sensor against the skin under operating conditions. This structure of the electrodes (10), (11), (12) defines a system stabilising the pressing force F (28). This pressure (defined by the pressing force F) is important for the quality of the performed measurements, however excessive long-term pressure can cause discomfort for a user during long-term wearing of the band. In order to minimise this effect, the electrodes (10, 11, 12) have been designed such that the ratio of their surface area to the inner surface area of the fastening strips (4) and (5) is lower than one. This provides minimisation of the compressing pressure P (29) of the band on the arm. Hence, for the required pressing force F (28), the compressing pressure P (29), experienced by the user of the band, is minimised. Compression P should be understood as a pressing force F acting on a unit area S, that is P=F/S. Additionally, the structure of the electrodes (10), (11), (12) as matrices (15), (50), (51), respectively, (FIG. 1, FIG. 3), comprised of movable sensors (16) provides, despite movement ±ΔL₁ of the band around and ±ΔL₂ along the arm (FIG. 2), measuring the ECG signal in a constant area on the arm, using adaptively changing configurations (27) (FIG. 4) of movable sensors (16) within a particular electrode (10), (11), (12). Number N of movable sensors (16) in each matrix (15), (50), (51) depends on the size of the electrode (10), the size of the movable sensor (16), and the dimensions of the matrix (15) itself. Therefore, if the geometrical dimensions of a single movable sensor (16) are known, the matrix (15) can comprise 1 to N of them. Nevertheless, a condition limiting the size of the electrode (10), (11), or (12) is the ratio of its surface area to the inner surface area of the fastening strips (4) or (5). Exemplary electrodes of a matrix of size of N=45 movable sensors are shown in FIG. 2B, FIG. 4. The configuration (27) of movable sensors (16) in each electrode (FIG. 4) is determined based on artificial intelligence algorithms using machine learning methods and neural networks. This provides stable impedance parameters on the interface between the electrodes (10, 11, 12) and the skin. These algorithms are a part of the microprocessor system software of the measurement module (1). The artificial intelligence algorithms for determining the configuration (27) of the movable sensors (16) in the matrix system (15), (50), (51) of the electrodes (10), (11), (12) stabilise the impedance value in the measurement area. ‘Configuration’ of k (27) movable sensors (16) should be understood as a system of n≤N movable sensors (16), which has been chosen in a particular electrode (10), (11), (12) to measure the ECG signal, which is shown in (FIG. 4) (configuration Z1 is comprised of n=19 movable sensors (16), configuration Z2 is comprised of n=16 movable sensors), where black indicates (active) movable sensors (16) belonging to the configuration Z1, Z2, and white indicates movable sensors (16) not belonging to the configuration. The scenario shown in (FIG. 4) can refer to any of the electrodes (10), (11), or (12). Adaptive changes of the configuration (27) of movable sensors (16) allow limiting the impedance changes and thus improve the quality of measured ECG signals. In each configuration (27) the number and arrangement of movable sensors (16) depends on the current impedance parameters on the electrode-skin interface. The number of active movable sensors (16) in the electrode can be equal to 1 to the maximum number of N in a particular electrode.

The band, after the power is turned on, begins a three-electrode measurement of the ECG signal using the electrodes (10), (11), and (12). It is performed by determining the electrical potential difference in a three-electrode system. The ECG signal measurement is performed on a constant area of the arm, despite the movement ±ΔL₁ of the band around or shifting ±ΔL₂ the band along the arm (FIGS. 2A-2B), using an adaptively changing configuration (27) of movable sensors (16). FIG. 2A illustrates a case of the band shifting around the arm, i.e., along its circumference, where black indicates the configuration (27) of movable sensors (16). The measurement area should be understood as an area of skin providing the ECG signal through the configuration (27) of movable sensors (16) within the electrode (10). Movement of the measurement area by a distance ±ΔL, as shown in FIG. 2A, causes a selection of a new configuration (27) (of other movable sensors (16)) in a corresponding electrode (10), (11), (12). This way, the electrodes ‘keep up’ with the moving measurement area and the ECG signal is measured on a constant area of contact with the skin. Similarly, the configuration (27) of movable sensors (16) within the electrodes (10), (11), (12) is adaptively adjusted in case of shifting the band along the arm. As a result, in case of a combination of longitudinal and transverse movement, the electrodes ‘keep up’ with the moving measurement area and the ECG signal is measured on a constant area of contact with the skin. FIG. 5 shows a block diagram of ECG signal processing and analysis. The ECG signal obtained from the electrodes (10), (11), and (12) is pre-filtered by an electronic component (30), and then it is converted from an analogue to a digital form in the ADC component (31) (FIG. 5). Next, the ECG signal processed by the ADC converter is processed by the software of the band. Signal sampling is a long-term ECG measurement, which is characterised in that the probability of detecting a cardiac activity anomaly is increased many times. The artefact detector (34), based on signal shape analysis, filters the artefacts from the resulting stream of ECG signal values. The artefact detection signal is included for building the personalised classifiers (32) and aggregating classifiers (33). A classifier should be understood as a variable category in the band software, which indicates an ECG signal belonging to a particular category of cardiac activity anomaly, wherein: a personalised classifier—adjusts the ECG signal classification criteria to the individual situation of the patient (e.g., physiological bradycardia in case of a person intensively practicing sports), an aggregating classifier—categorizes individual fragments of the ECG signal into individual anomaly groups (e.g., based on morphology of the QRS complexes). Both types of classifiers are completely independent types of ECG signal processing. The classifiers work simultaneously with the artefact detector. The artefact detector (34) provides information for the classifiers (32), (33) illustrated in FIG. 5 below and above the detector (34). Personalised classifiers (32) characteristic for a particular person, in a period without anomalies (heart rate characteristics during rest and during exertion), are recognised and stored in the band. The ECG signal analysis is performed taking into account individual characteristics of the user, rather than average values for the population. This provides individualised and more reliable assessment of ECG recording for a particular user. The aggregating classifiers (33) aggregate information carried by the ECG signal, while simultaneously reducing the number of stored samples. The signal is then transmitted to the anomaly selector (35), where the ECG signal reading occurs selectively based on personalised (32) and aggregating (33) classifiers, and fragments of the ECG recording are extracted, which may contain information on cardiac activity anomalies. In this way, technology is made available to support the assessment of the user's health by a doctor. The anomaly selector (35) extracts cardiac activity abnormalities, i.e., distinguishes the artefacts from an actual absence of QRS complexes and detects: sudden increases and decreases in heart rate, occurrence of R-R intervals variability or its absence. Cardiac activity anomalies should be understood as the following, outside the range of accepted normal values: increases and decreases of heart rate frequency (normal range of: 50-100/min), occurrence of R-R intervals variability (number of cardiac activity cycles differing by more than 20 ms with respect to a previous cycle) or its absence.

1. An arm-worn electrocardiographic device in a shape of a band, comprising a measurement module for processing the measured electrocardiographic signal, characterised in that the first measuring wing (2) and the second measuring wing (3) are attached to the measurement module (1), symmetrically with respect to each other, by means of spring connectors (7, 8), for measuring the electrocardiographic signal, and a first fastening strip (4) and a second fastening strip (5) are attached to each of the measuring wings (2, 3), respectively, by means of spring connectors (6, 9), wherein the module (1), the measuring wings (2, 3), and the fastening strips (4, 5) have an inner surface for contacting the user's skin and an outer surface for attaching the personalizing attachment (26), and on the inner surface of the module (1) a third electrode (10) is located, on the inner surface of the first measuring wing a first second electrode (11) is located, and on the inner surface of the second measuring wing (5) a second third electrode (12) is located, wherein each of the electrodes (10, 11, 12) comprises a matrix (15, 50, 51) of movable sensors (16), respectively, wherein the surface area ratio of each electrode (10, 11, 12) to the surface area of the inner surface of the first fastening strip (4) or the second fastening strip (5) is not greater than one, wherein the movable sensor is comprised of a conductive material (16a) bonded to an elastic substrate (14).

2. The arm-worn electrocardiographic device according to claim 1, characterised in that each of the matrices (15, 50, 51) is comprised of N movable sensors (16).

3. The arm-worn electrocardiographic device according to claim 1 or 2, characterised in that, in the movable sensor (16), the elastic substrate (14) adheres to the conductive material (16a) on the opposite surface of the material (16a) with respect to its surface configured for contact with the skin.

4. The arm-worn electrocardiographic device according to claims 1 to 3, characterised in that the elastic substrate (14) of the movable sensor (16) is an elastomer.

5. The arm-worn electrocardiographic device according to claim 2 or 3, characterised in that the conductive material (16a) of the movable sensor (16) is chosen from a group including: a metal or a conductive plastic.

6. The arm-worn electrocardiographic device according to claim 1, characterised in that the first (2) and second (3) measuring wings comprise power batteries (17) and (18), wherein the first (2) or the second (3) measuring wing comprises a magnetic socket (13) for attaching an electrical connector and maintaining mechanical stability of the electrical connection with the device for battery (17, 18) charging, for a proper connection between the plug (25) and the socket (13).

7. The arm-worn electrocardiographic device according to claim 1, characterised in that the measurement module (1) for processing the measured electrocardiographic signal comprises a component for measuring the electrocardiographic signal, a microprocessor component comprising an analogue-to-digital converter, and a component for wireless communication with an external device.

8. The arm-worn electrocardiographic device according to claims 1 to 7, characterised in that the matrix (15, 50, or 51) of movable sensors in an arrangement of k columns and l rows of movable sensors (16) comprises 1 to N movable sensors (16).

9. A method for electrocardiographic signal measurement by means of the arm-worn electrocardiographic device, as defined in claim 1, including:
a) placing the device on the arm,
b) measuring the electrocardiographic signal, where the electrocardiographic signal measurement is achieved by determining the electrical potential difference in a three-electrode system,
c) processing and analysing the measurement signal in order to identify the features characteristic of cardiac activity,
characterised in that the single electrode in the three-electrode system corresponds to any electrode (10, 11, 12) of the arm-worn electrocardiographic device, wherein, in order to obtain the electrical potential difference, a first configuration of the movable sensors (16) in the matrix arrangement (15, 50, 51) of the electrodes (10, 11, 12) is determined by means of a system of a microprocessor measuring module (1), and a measurement of the electrical potential difference is performed, wherein, if the measurement area corresponding to the selected electrode (10, 11, or 12) undergoes shifting during the measurement, then a subsequent configuration of the movable sensors (16) in the matrix arrangement (15, 50, 51) of the electrodes (10, 11, 12) is determined by the microprocessor system of the measurement module (1), wherein the movable sensor (16) is a conductive material (16a) bonded with the elastic substrate (14), and the measurement of the electrical potential difference is performed, wherein, throughout the measurement, K configurations of movable sensors (16) in a matrix arrangement (15, 50, 51) of the electrodes (10, 11, 12) are determined, in order to provide the electrocardiographic signal, in such a way that the value of impedance in the measurement area corresponding to the subsequent configurations of the movable sensors (16) in the matrix arrangement (15, 50, 51) of the electrode (10, 11, or 12) is stable.

10. The method according to claim 9, characterised in that the matrix (15, 50, or 51) of movable sensors in an arrangement of k columns and l rows of movable sensors (16) comprises 1 to N movable sensors (16).

11. The method according to claim 9 or 10, characterised in that the configuration of the movable sensors (16) in a matrix arrangement (15, 50, 51) of floating electrodes (10, 11, 12) comprises 1 to N movable sensors (16), where N corresponds to a maximum number of movable sensors (16) in a particular electrode (10, 11, or 12).

12. The method according to any of the claims 9 to 11, characterised in that the configuration of movable sensors (16) is determined by the microprocessor system of the measurement module (1) using artificial intelligence algorithms, wherein the configuration of movable sensors in a matrix arrangement (15, 50, 51) of the electrodes (10, 11, 12) is determined based on impedance changes in the measurement area.

13. The method according to claim 9, characterised in that the ECG measurement signal is processed in an analogue-to-digital converter (31) and analysed using electrocardiographic software of the arm-worn device, where the software includes an aggregating classifier (33) system for categorising fragments of the electrocardiographic signal, a personalised classifier (32) for classifying the criteria of electrocardiographic signal classification, an artefact detector (34) for filtering out the artefacts from the electrocardiographic signal, and an arrhythmia selector (35) for extracting the features characteristic for cardiac activity based on the aggregating and personalised classifiers, preferably cardiac activity abnormalities which include the following, outside the range of accepted normal values: increases and decreases in heart rate, occurrence of variability of R-R intervals or its absence.

14. The method according to claim 9 or 13, characterised in that the ECG signal measurement is performed by an ECG measuring component and sent to the ADC converter, the signal is sent to the artefact detector (34) for identification and filtering out the artefacts, next, independently with respect to each other, based on the detected artefact signal, the personalised classifiers and aggregating classifiers are built in the personalised classifier (32) and in the aggregating classifier (33), respectively, next, the signal is sent to the arrhythmia selector (35) and the cardiac activity anomalies are extracted from it based on the personalised classifiers (32) and aggregating classifiers (33), and are transmitted to a mobile application by the component for wireless communication with an external device of the measurement module (1).
